(19) United States
(12) Patent Application Publication
(10) Pub. No.: US 2026/0198788 A1
(43) Pub. Date: Jul. 16, 2026

(54) SYSTEM, STORAGE MEDIUM, AND APPARATUS

(71) Applicant: TERUMO KABUSHIKI KAISHA, Tokyo (JP)

(72) Inventors: Shinsuke MATSUMOTO, Sagamihara Kanagawa (JP); Taiki HAYASHI, Ota Tokyo (JP); Yuto MASUDA, Hiratsuka Kanagawa (JP)

(21) Appl. No.: 19/565,469

(22) Filed: Mar. 12, 2026

Related U.S. Application Data
(63) Continuation of application No. PCT/JP2024/032399, filed on Sep. 10, 2024.

(30) Foreign Application Priority Data
Sep. 14, 2023 (JP) 2023-149393

(51) Int. Cl. A61B 5/0205; A61B 5/00; A61B 5/11

(52) U.S. Cl. CPC A61B 5/0205; A61B 5/1118; A61B 5/6802; A61B 5/7275

(57) ABSTRACT

A system for determining heart failure exacerbation includes a wearable device measuring biological data and motion data of a subject, and a server connectable to the wearable device to: acquire the biological data and motion data at each time point in a predetermined period; calculate a parameter related to a heart based on the biological data; calculate an activity amount based on the motion data; identify active time points where the calculated activity amounts are equal to or greater than a predetermined value; identify specific parameters at delayed time points when a predetermined time has elapsed from the active time points; and calculate an index value related to the heart failure exacerbation based on a relationship between the activity amounts at the active time points and the specific parameters at the delayed time points.

### CROSS-REFERENCE TO RELATED APPLICATION

This application is a continuation of International Patent Application No. PCT/JP2024/032399 filed Sep. 10, 2024, which is based upon and claims the benefit of priority from Japanese Patent Application No. 2023-149393, filed Sep. 14, 2023, the entire contents of which are incorporated herein by reference.

### BACKGROUND

### Technical Field

The present disclosure relates to a program, an information processing method, and an information processing apparatus.

### Related Art

Various methods for supporting determinations related to the clinical status (e.g., exacerbation) of heart failure have been proposed. For example, there is a known device for determining the state (i.e., clinical status) of congestive heart failure based on physiological values such as bioimpedance, electrocardiogram (ECG), and 3-axis acceleration.

### SUMMARY

Generally, in processing physiological information, it is technically difficult for a computer to distinguish between changes caused by the exacerbation of a condition and changes caused by daily physical movements or long-term trends. The present disclosure addresses this issue to provide an improvement to computer-based determination technology. By analyzing biological data in association with motion data rather than analyzing biological data in isolation, the information processing according to the present disclosure enables a computer to more accurately extract features reflecting the clinical status of heart failure, thereby enhancing the precision of the calculated index value.

One embodiment provides a system for determining heart failure exacerbation, comprising: a wearable device including one or more sensors configured to measure biological data and motion data of a subject; and a server connectable to the wearable device and including a processor and a memory storing a program that, when executed by the processor, causes the server to: acquire, from the wearable device, the biological data and the motion data at each time point in a predetermined period, calculate a parameter related to a heart at each time point based on the biological data, calculate an activity amount of the subject at each time point based on the motion data, identify active time points among time points in the predetermined period where the calculated activity amounts are equal to or greater than a predetermined value, identify specific parameters at delayed time points when a predetermined time has elapsed from the identified active time points, and calculate an index value related to the heart failure exacerbation based on a relationship between the activity amounts at the active time points and the specific parameters at the delayed time points.

### BRIEF DESCRIPTION OF DRAWINGS

FIG. 1 is an explanatory diagram illustrating a configuration of a heart failure exacerbation determination system.

FIG. 2 is a block diagram illustrating a configuration example of a server.

FIG. 3 is a block diagram illustrating a configuration example of a wearable device.

FIG. 4 is an explanatory diagram related to a first index value.

FIG. 5 is an explanatory diagram related to a second index value.

FIG. 6 is an explanatory diagram related to a third index value.

FIG. 7A is an explanatory diagram related to the third index value.

FIG. 7B is an explanatory diagram related to the third index value.

FIG. 8 is an explanatory diagram related to a fourth index value.

FIG. 9 is an explanatory diagram related to a fifth index value.

FIG. 10 is an explanatory diagram related to the fifth index value.

FIG. 11 is an explanatory diagram related to the fifth index value.

FIG. 12 is a flowchart illustrating an example of a process executed by a server.

FIG. 13 is a flowchart illustrating an example of a process executed by a server according to a modification.

### DETAILED DESCRIPTION

Hereinafter, embodiments of the present disclosure will be described in detail based on the accompanying drawings.

### Embodiment 1

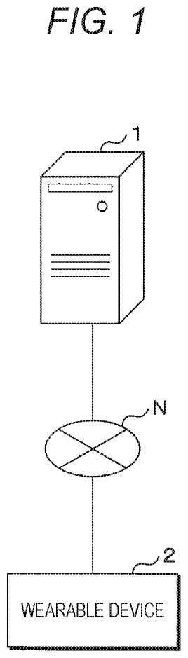

FIG. 1 is an explanatory diagram illustrating a configuration of a heart failure exacerbation determination system. The present embodiment will describe a heart failure exacerbation determination system that calculates an index value related to exacerbation of heart failure from biological data and motion data of a subject. The heart failure exacerbation determination system includes a server 1 and a wearable device 2. The devices are communicably connected via a network N such as the Internet.

The server 1 is a server computer or apparatus capable of performing various types of information processing, and transmitting and receiving information, and analyzes biological data and motion data of a subject (i.e., patient) measured by the wearable device 2. As will be described in detail later, the server 1 calculates a parameter (e.g., pulse rate) related to the heart of the subject from the biological data of the subject, and also calculates the activity amount of the subject from the motion data. Then, the server 1 calculates an index value related to exacerbation of heart failure of the subject based on the calculated parameter and activity amount.

The wearable device 2 is a portable device worn on a wrist or the like of the subject, and measures biological data and motion data of the subject. Specifically, the wearable device 2 measures a photoplethysmography (PPG) signal as the biological data, and measures acceleration as the motion data. For example, the wearable device 2 includes a light emitting diode (LED) light source and a light receiving element for the LED light, and measures a PPG signal by emitting an infrared light or a green LED light to the skin of the subject, and then receiving a reflected light of the LED light. In addition, the wearable device 2 incorporates an acceleration sensor, and measures acceleration at the time of PPG measurement.

The present embodiment is described based on the assumption that PPG is measured as the biological data, but the present embodiment is not limited thereto. For example, it is also possible to measure ECG, respiration, blood pressure, and the like as the biological data, and calculate a parameter related to the heart (e.g., heart rate, heart rate variability, and the like for the ECG; respiration rate, respiration complexity, and the like for the respiration; and arterial pressure, venous pressure, and the like for the blood pressure) from such data.

The present embodiment is described based on the assumption that acceleration is measured as the motion data, but the present embodiment is not limited thereto. For example, it is also possible to use an angular velocity measured with a gyro sensor as the motion data, and calculate the activity amount of the subject from the angular velocity.

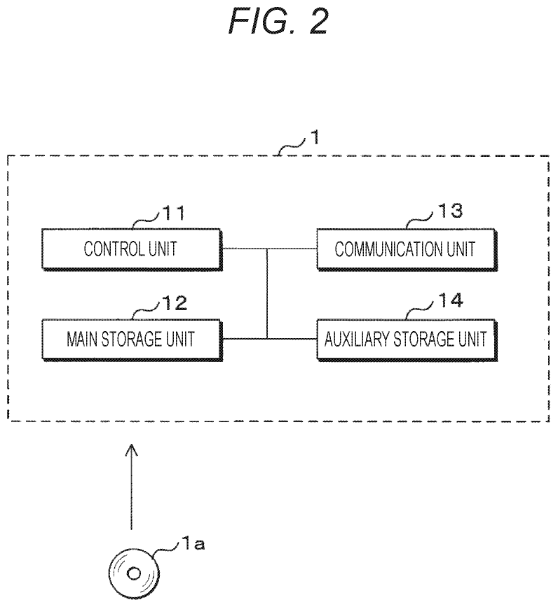

FIG. 2 is a block diagram illustrating a configuration example of the server 1. The server 1 includes a control unit 11 and one or more arithmetic processing units, such as central processing units (CPUs), micro-processing units (MPUs), and graphics processing units (GPUs), and performs various types of information processing, control processes, and the like by reading and executing programs stored in an auxiliary storage unit 14. A main storage unit 12 is a temporary storage area, such as a static random access memory (SRAM) or a dynamic random access memory (DRAM), and temporarily stores data necessary for the control unit 11 to execute arithmetic processing. A communication unit 13 is a communication module or network interface circuit for performing a process related to communication, and transmits and receives information to and from the outside. The auxiliary storage unit 14 is a non-volatile storage area, such as a high-capacity memory or a hard disk, and stores programs (i.e., program products) necessary for the control unit 11 to execute processes, and other data.

Note that the auxiliary storage unit 14 may be an external storage device connected to the server 1. In addition, the server 1 may be a multicomputer including a plurality of computers, or may be a virtual machine virtually constructed with software.

In the present embodiment, the configuration of the server 1 is not limited to that described above, and the server 1 may include an input unit that receives a user input, and a display unit that displays an image, for example. In addition, the server 1 may include a reading unit that reads a portable storage medium 1a, such as a compact disk (CD)-ROM or a digital versatile disc (DVD)-ROM, and may read and execute a program from the portable storage medium 1a.

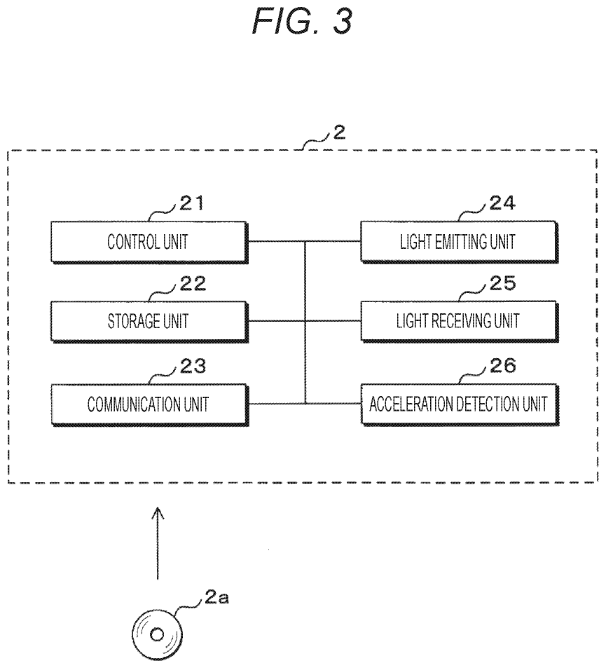

FIG. 3 is a block diagram illustrating a configuration example of the wearable device 2. The wearable device 2 includes a control unit 21, a storage unit 22, a communication unit 23, a light emitting unit 24, a light receiving unit 25, and an acceleration detection unit 26.

The control unit 21 includes one or more processors, such as CPUs, and performs various types of information processing by reading and executing programs stored in the storage unit 22. The storage unit 22 is a memory, such as a RAM or a ROM, and stores programs (i.e., program products) necessary for the control unit 21 to execute processes, and other data. The communication unit 23 is a communication module for performing a process related to communication, and transmits and receives information to and from the outside. The light emitting unit 24 is an LED light source, and emits an LED light. The light receiving unit 25 is a light receiving element that receives a reflected light reflected from the skin of the subject. The acceleration detection unit 26 is an acceleration sensor, and detects the acceleration of the subject based on a motion of the subject.

Note that the wearable device 2 may include a reading unit that reads a portable storage medium 2a, such as a CD-ROM, and may read and execute a program from the portable storage medium 2a.

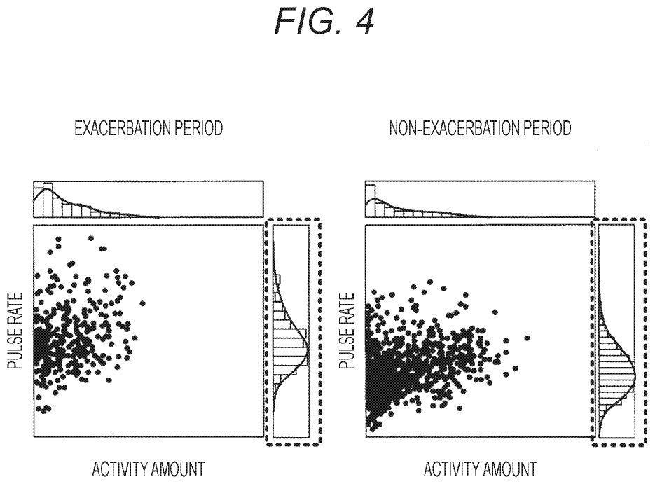

FIG. 4 is an explanatory diagram related to a first index value. FIG. 4 illustrates a scatter diagram in which a parameter (i.e., pulse rate) related to the heart calculated from the biological data (i.e., PPG signal) and the activity amount of the subject calculated from the motion data (i.e., acceleration) are plotted. A summary of the present embodiment will be described below.

As described above, the server 1 acquires biological data and motion data of a subject from the wearable device 2, and calculates an index value related to exacerbation of heart failure from the biological data and the motion data. For example, the server 1 periodically communicates with the wearable device 2 to acquire biological data and motion data at each time point in a predetermined period.

The server 1 calculates a parameter related to the heart at each time point in the measurement period based on the acquired biological data. Specifically, the server 1 acquires a PPG signal as the biological data, and calculates a pulse rate from the PPG signal.

In addition, the server 1 calculates the activity amount (i.e., amount of physical activity or activity level) of the subject at each time point in the measurement period based on the motion data acquired from the wearable device 2. For example, the server 1 acquires acceleration as the motion data, and calculates a numerical value related to the activity amount from the acceleration according to Expression (1) below.

$$\begin{matrix}[ Math . 1 ] & \\ Activity = \sqrt{{x}^{2} + {y}^{2} + {z}^{2}} - g & ( 1 )\end{matrix}$$

Note that x, y, and z denote accelerations in the x-axis direction, the y-axis direction, and the z-axis direction, respectively, and g denotes gravitational acceleration. For example, the server 1 counts, as activity intensity, the number of times the numerical value calculated with Expression (1) exceeds a predetermined value within a predetermined time over time, and calculates, as the activity amount, the moving average of the activity intensity obtained as time-series data within a predetermined time window width. Herein, determining the moving average of the activity intensity as the activity amount makes it possible to suppress the influence of an instantaneous motion, such as swinging of an arm, and to determine the activity amount that more accurately reflects systemic activity that puts stress on the heart.

The server 1 calculates an index value related to exacerbation of heart failure based on the parameter and activity amount calculated as described above. In the present embodiment, first to fifth index values described below are calculated as the index value.

First, the content of a process performed to calculate the first index value will be described. FIG. 4 illustrates a scatter diagram for each of an exacerbation period and a non-exacerbation period, in which the activity amount and the parameter after a predetermined time (e.g., one hour) has elapsed from a measurement time point of the activity amount are plotted based on data at a plurality of time points when the activity amount is greater than or equal to a given value. The horizontal axis of the scatter diagram represents the activity amount, and the vertical axis represents the parameter (i.e., pulse rate).

When the two scatter diagrams are compared, it is seen that, as indicated by rectangular dashed lines in FIG. 4, a distribution of the parameter in the exacerbation period often has a higher value than a distribution of the parameter in the non-exacerbation period. Therefore, it can be said that the distribution of the parameter based on the data obtained for a predetermined period (e.g., two weeks) reflects the state (i.e., clinical status) of heart failure of the subject in that period. Thus, the server 1 calculates the first index value as follows, thereby calculating a difference in the parameter distribution as an index.

First, the server 1 calculates, from biological data and motion data in each of a past period (i.e., first period) and the most recent period (i.e., second period), the parameter and the activity amount at each time point in each of the past period and the most recent period. The past period is, for example, a period from the current time point back to a given period earlier (i.e., six months earlier). The most recent period is, for example, a period (e.g., past two weeks) from the current time point back to a given period earlier. In addition, the past period (i.e., first period) is a period during which a medical professional confirms in advance that the subject's heart failure has been in a non-exacerbation period or an exacerbation period. By storing a parameter indicating the state of the heart and the activity amount for that period in the server 1, it is possible to allow the past period (i.e., first period) to serve as a reference for calculating an index value in comparison with the most recent period (i.e., second period).

For each of the past period and the most recent period, the server 1 identifies each time point when the activity amount is greater than or equal to a given value (hereinafter referred to as an “active time point”), and identifies a parameter (i.e., pulse rate) at each time point when a predetermined time (e.g., one hour) has elapsed from each identified active time point. Accordingly, the server 1 generates distribution information on a distribution of the parameter after a predetermined time has elapsed from an active time point when the activity amount is greater than or equal to the given value.

The reason why the parameter at a time point when a predetermined time has elapsed from an active time point is used instead of directly using the parameter at the active time point is that the heart rate does not increase significantly immediately after vigorous movement, and the heart rate does not decrease easily even after the lapse of time, because a patient with chronic heart failure has a reduced cardiac function. By calculating the index value based on the activity amount at an active time point and a parameter at a time point when a predetermined time has elapsed from a measurement time point of the activity amount, it is possible to suitably grasp exacerbation of heart failure in a patient with chronic heart failure.

The server 1 calculates the first index value by comparing the parameter distribution information for the past period with the parameter distribution information for the most recent period. For example, the server 1 calculates Kullback-Leibler divergence (KL divergence; KL information volume) as a statistic that quantifies the difference between the two distributions. Accordingly, the server 1 calculates the difference between the distributions of the parameters as the index.

Although the KL divergence is calculated as the first index value in the above description, the present embodiment is not limited thereto. For example, the server 1 may calculate, as the first index value, a basic statistic (i.e., first statistic), such as a median or a mode indicating a distribution, based on a distribution of the parameter at a time point when a predetermined time has elapsed from a measurement time point of the activity amount at an active time point. With such a statistic, it is also possible to present, as the index, how high the parameter (i.e., pulse rate) is, that is, an increase in the parameter due to exacerbation of heart failure.

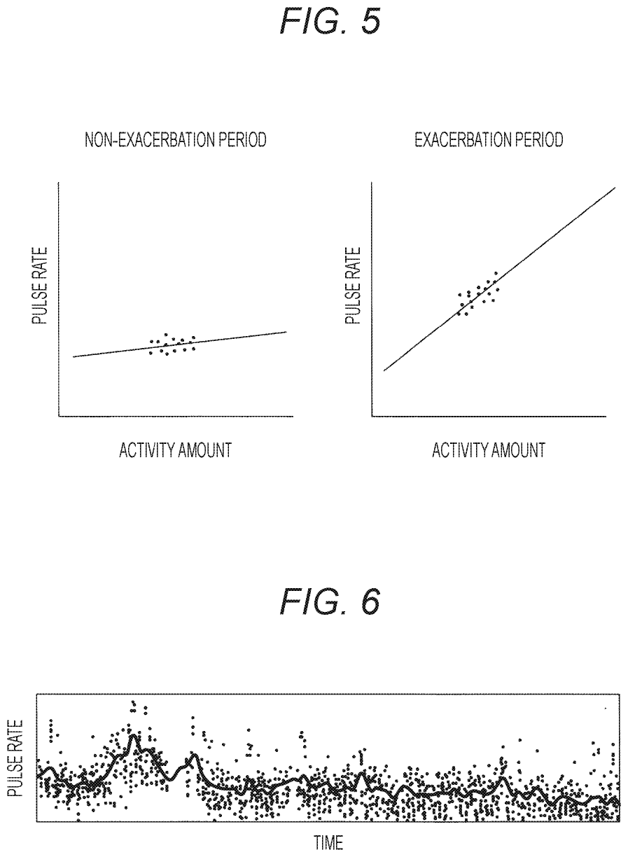

FIG. 5 is an explanatory diagram related to the second index value. FIG. 5 illustrates a scatter diagram in which the activity amount and the parameter (i.e., pulse rate) are plotted in a case where the activity amount is greater than or equal to a given value.

FIG. 5 illustrates a scatter diagram for each of an exacerbation period and a non-exacerbation period, in which the activity amount and the parameter (i.e., pulse rate) after a predetermined time (e.g., 30 minutes) has elapsed from a measurement time point of the activity amount are plotted. Here, regarding the case where the activity amount is greater than or equal to a given value, when a regression line of the activity amount and the parameter is derived, it is found that a slope of the regression line for the exacerbation period is steeper overall. Therefore, it can be said that the slope of the regression line derived from the data obtained over a predetermined period (e.g., two weeks) reflects the state of heart failure of the subject in that period.

Thus, the server 1 calculates the second index value based on the slope of the regression line. First, the server 1 calculates the parameter and the activity amount from biological data and motion data for the most recent period (e.g., the past two weeks). The server 1 derives, for the most recent period, a regression line indicating a relationship between the activity amount and the parameter based on distributions (i.e., scatter diagrams) of the activity amount at an active time point where the activity amount is greater than or equal to a given value and the parameter at a time point when a predetermined time has elapsed from a measurement time point of the activity amount. The server 1 calculates the slope of the derived regression line as the second index value.

Although the slope of the regression line is calculated as the second index value in the above description, the present embodiment is not limited thereto. For example, when determining distributions of the activity amount and the parameter, the server 1 may determine a plurality of distributions by identifying parameters at a plurality of time points, such as 10 minutes, 30 minutes, and 60 minutes, etc., having elapsed from a measurement time point of the activity amount, and derive regression lines from the respective distributions so as to calculate, as the second index value, the average value, the total value, or the like of slopes of the regression lines. In this manner, the server 1 only needs to be able to calculate the second index value based on the slope(s) of the regression line(s).

In the above description, the second index value is calculated only from data for the most recent period, but the present embodiment is not limited thereto, and the second index value may be calculated by comparing data for the most recent period with data for the past period (e.g., half a year ago). That is, the server 1 calculates a slope of a regression line by determining a distribution of the parameter with respect to the activity amount from biological data and motion data for the most recent period, and calculates a slope of a regression line by determining a distribution of the parameter with respect to the activity amount from biological data and motion data for the past period, and then calculates, as the second index value, the difference, the ratio, or the like between the two slopes. In this manner, the server 1 may calculate the second index value based on the slope for the most recent period and the slope for the past period.

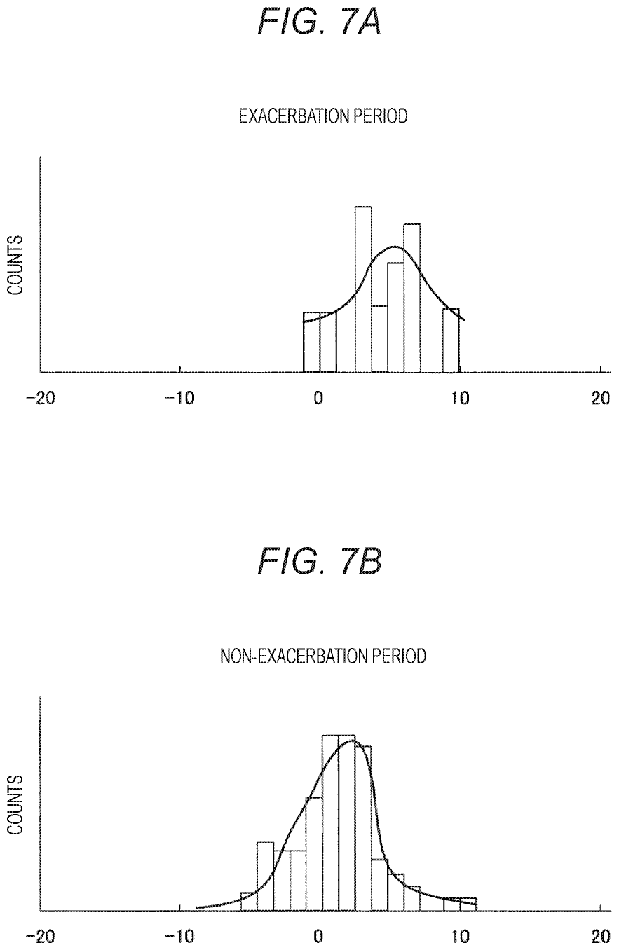

FIGS. 6, 7A, and 7B are explanatory diagrams related to the third index value. FIG. 6 is a graph illustrating the parameter (i.e., pulse rate) and a moving average waveform of the parameter. FIGS. 7A and 7B are histograms respectively illustrating distributions of difference values between the parameter and the moving average for an exacerbation period (FIG. 7A) and a non-exacerbation period (FIG. 7B).

The server 1 calculates the third index value based on a distribution of the parameter at an active time point when the activity amount is greater than or equal to a given value, as with the first index value and the second index value. Here, when data is divided into that of the exacerbation period and that of the non-exacerbation period, it is desirable to observe the relationship of the parameter with the activity amount, but there may be a case where the influence of the long-term trend of the parameter and the influence of the activity amount cannot be distinguished (see FIG. 6).

Therefore, the server 1 calculates a difference value between the parameter and the moving average of the parameter. This eliminates the influence of the long-term trend.

FIGS. 7A and 7B are histograms respectively illustrating distributions of the difference values for the exacerbation period and the non-exacerbation period, in a case where the activity amount is greater than or equal to a given value. As illustrated in FIGS. 7A and 7B, in the case where the activity amount is greater than or equal to a given value, the histogram for the exacerbation period is shifted to the right, indicating larger difference values. Therefore, it can be said that a distribution of the difference value for a predetermined period (e.g., two weeks) reflects the state of heart failure of the subject in that period.

Thus, the server 1 calculates the difference between distributions in the histograms as the third index value. First, the server 1 calculates the parameter and the activity amount from biological data and motion data for each of a past period (i.e., first period) and the most recent period (i.e., second period), and calculates a difference value between the parameter at each time point and the moving average of the parameter for each of the past period and the most recent period. For each of the past period and the most recent period, the server 1 generates difference value distribution information on a distribution of the difference value at an active time point when the activity amount is greater than or equal to a given value. Then, the server 1 calculates the third index value by comparing the difference value distribution information for the past period with the difference value distribution information for the most recent period. Specifically, as in the case of calculating the first index value, the server 1 calculates, as the third index value, the KL divergence, which is a statistic that quantifies the difference between the distributions.

Although the KL divergence is calculated as the third index value in the above description, the present embodiment is not limited thereto. For example, the server 1 may calculate, as the index value, a basic statistic (i.e., second statistic) such as a median or a mode indicating a distribution of the difference value at an active time point. A shift of the histogram due to exacerbation of heart failure can also be determined by using the statistic. In addition, to generate the difference value distribution information, it is also possible to, instead of calculating the difference value at each time point, identify an active time point when the activity amount is greater than or equal to a given value, and calculate the difference value only at the identified active time point.

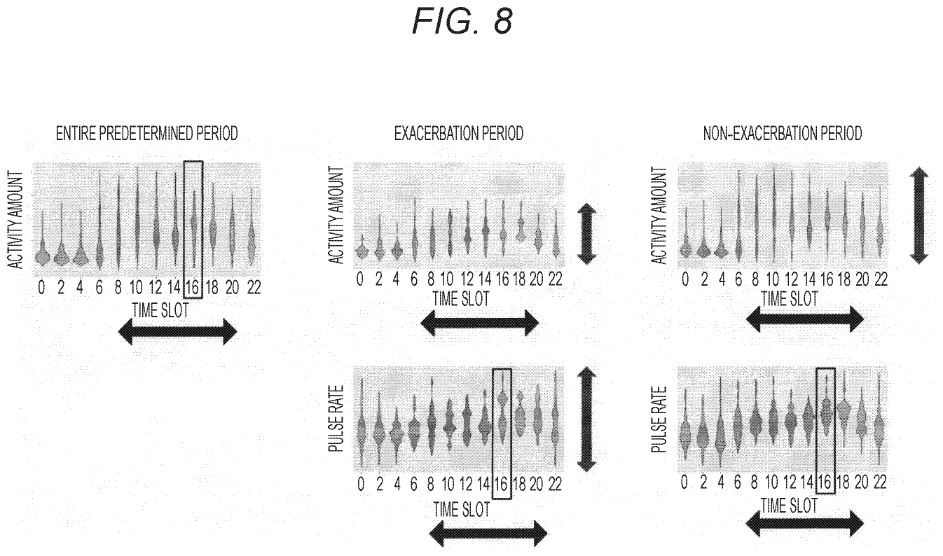

FIG. 8 is an explanatory diagram related to the fourth index value. FIG. 8 illustrates a violin plot representing a distribution of the activity amount for each time slot (e.g., each time slot obtained by dividing a day into two-hour intervals) in a predetermined period, and a violin plot representing each of distributions of the activity amount and the parameter (i.e., pulse rate) for each time slot (e.g., each time slot obtained by dividing a day into two-hour intervals) in each of an exacerbation period and a non-exacerbation period of the predetermined period.

As illustrated in FIG. 8, a distribution of the activity amount at 16:00 (4:00 PM) is biased to the higher side as compared with the other time slots, and thus, it can be said that the subject is most active during this time slot of the day. During such a time slot, a distribution of the parameter (i.e., pulse rate) in the exacerbation period is higher than that in the non-exacerbation period. Therefore, it can be said that the distribution of the parameter for the time slot reflects the state of heart failure of the subject. Thus, the server 1 calculates the fourth index value from the distributions of the activity amount and the parameter for each time slot.

First, the server 1 calculates the activity amount from motion data for a predetermined period, such as a past period (e.g., half a year ago) or the most recent period (e.g., the past two weeks), and identifies a distribution of the activity amount for each time slot. From the identified distribution of the activity amount for each time slot, the server 1 identifies an active time slot for which a statistic (e.g., median or mode) indicating a distribution of the activity amount is greater than or equal to a given value. Next, the server 1 calculates the parameter from biological data for the most recent period (e.g., the past two weeks), and also identifies a distribution of the parameter for the active time slot. Then, the server 1 calculates, as the fourth index value, a statistic, such as a variance or a mode, indicating a distribution of the parameter for the active time slot.

In the above description, the fourth index value is calculated only from data for the most recent period, but the present embodiment is not limited thereto, and the fourth index value may be calculated by comparing data for the past period with data for the most recent period. Specifically, for each of the past period (e.g., half a year ago) and the most recent period, the server 1 calculates a statistic indicating a distribution of the parameter for the active time slot (e.g., 16:00 (4:00 PM)) identified based on the motion data for the predetermined period. Then, the server 1 calculates the fourth index value by, for example, determining the difference between the statistic of the parameter for the past period and the statistic of the parameter for the most recent period. Alternatively, the server 1 may calculate the fourth index value (e.g., KL divergence as a statistic that quantifies the difference between the distributions) by comparing the parameter distribution information for the active time slot of the past period with the parameter distribution information for the active time slot of the most recent period. In this manner, the server 1 may calculate the fourth index value by comparing data for the past period with data for the most recent period.

In addition, as illustrated in FIG. 8, during a time slot when the subject is active, such as from 8:00 AM to 20:00 (8:00 PM), the parameter (i.e., pulse rate) is higher overall although the activity amount tends to be lower overall in the exacerbation period. Therefore, it can be said that the distributions of the activity amount and the parameter for the time slot reflect the state of heart failure of the subject. Thus, the server 1 may calculate the fourth index value by comparing data for the past period with data for the most recent period regarding the distributions of the activity amount and the parameter for each time slot. Specifically, for each of a past period (e.g., half a year ago) and the most recent period, the server 1 identifies the distributions of the activity amount and the parameter calculated from biological data and motion data, for the active time slot (e.g., from 8:00 AM to 20:00 (8:00 PM)) identified based on motion data for a predetermined period. Then, the server 1 calculates, for each of the distribution of the activity amount and the distribution of the parameter for the active time slot of each of the past period and the most recent period, KL divergence as a statistic that quantifies the difference between the distributions, and sums the calculated values to obtain the fourth index value. Note that each of the distribution of the activity amount and the distribution of the parameter for each of the past period and the most recent period may indicate the distribution for the entire duration (e.g., 12 hours) of the active time slot, and the KL divergence may be calculated using such a distribution. Alternatively, as illustrated in FIG. 8, each of the distribution of the activity amount and the distribution of the parameter for each of the past period and the most recent period may indicate the distribution for the active time slot divided into predetermined time intervals (e.g., two hours), and the fourth index value may be calculated by calculating the KL divergence for each time interval, and then summing them. In this specific example, the “given value of the statistic (e.g., median or mode) indicating the distribution of the activity amount” used to identify the active time slot may be a value lower than the value in the specific example described above, and may be a value higher than a statistic indicating a distribution of the activity amount for a sleeping time slot (for example, from 22:00 (10:00 PM) to 6:00 AM).

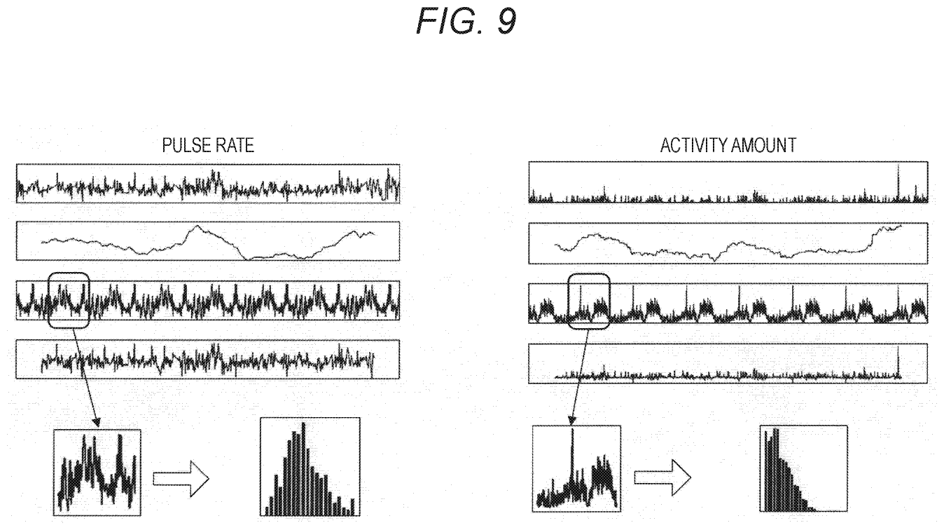

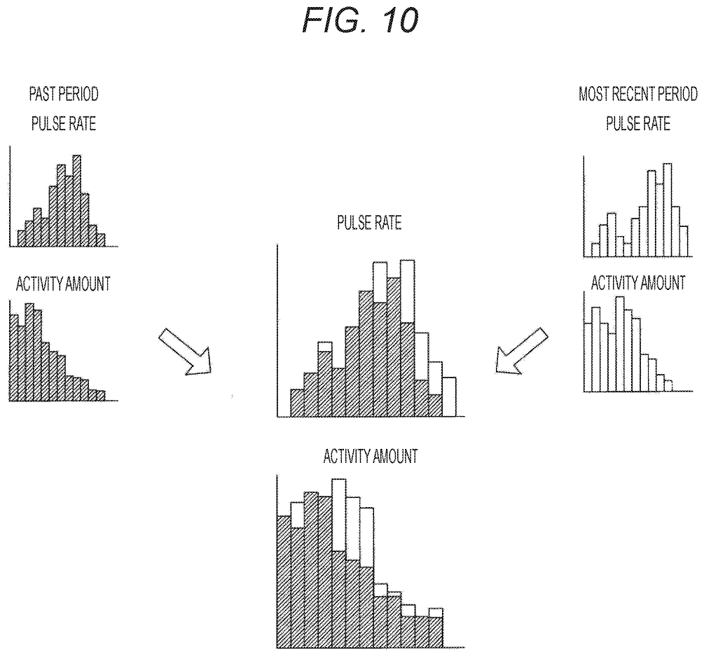

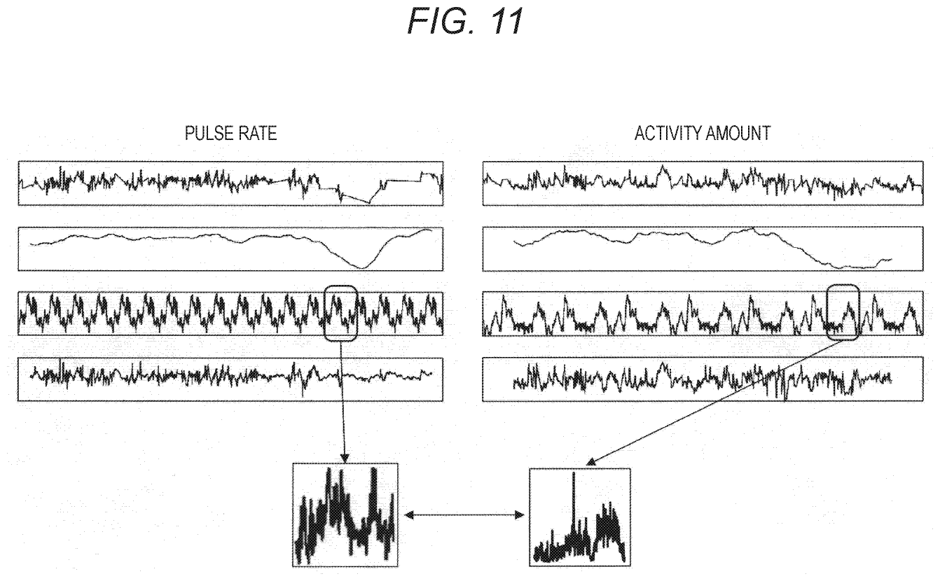

FIGS. 9 to 11 are explanatory diagrams related to the fifth index value. FIG. 9 illustrates a state in which original data (i.e., Observed) for each of the parameter (i.e., pulse rate) and the activity amount is separated into trend components (i.e., Trend), periodic components (i.e., Seasonal), and residual components (i.e., Residual), and periodic components for the same segment are extracted to generate a histogram. FIG. 10 illustrates a state in which a histogram of the past period (illustrated as a hatched histogram) is compared with a histogram of the most recent period (illustrated as an unfilled histogram). As with FIG. 9, FIG. 11 illustrates a state in which original data of each of the parameter and the activity amount is separated into trend components, periodic components, and residual components, and a correlation coefficient is calculated by comparing periodic components for the same segment.

The server 1 calculates the fifth index value by comparing periodic components of each of the parameter and the activity amount for a past period (i.e., first period) and the most recent period (i.e., second period). First, the server 1 calculates the parameter and the activity amount from biological data and motion data for each of the past period and the most recent period. As illustrated in FIG. 9, the server 1 separates each of the parameter and the activity amount (i.e., the first graph from the top) into trend components (i.e., the second graph from the top), periodic components (i.e., the third graph from the top), and residual components (i.e., the fourth graph from the top) for each of the past period and the most recent period, using the Seasonal-Trend decomposition using LOESS (STL) method, for example.

As illustrated in FIG. 9, the server 1 extracts, for each of the past period and the most recent period, periodic components for the same segment from each of the periodic components of the parameter and the activity amount. Data on the extracted time-series periodic components is converted into a histogram representing a distribution of the data. Note that when the periodic components are components based on a daily cycle, the same segment refers to a segment for one day (24 hours), for example. In this manner, the same segment may be matched with the period based on which the periodic components are separated from the original data. In addition, the same segment may be a segment for half a day (i.e., 12 hours) from 6:00 AM to 6:00 PM, for example.

As illustrated in FIG. 10, the server 1 calculates the fifth index value by comparing the extracted periodic components of each of the parameter and the activity amount between the past period and the most recent period. For example, the server 1 calculates, for each of the distribution (i.e., histogram) of the periodic components of the parameter and the distribution of the periodic components of the activity amount for each of the past period and the most recent period, KL divergence as a statistic that quantifies the difference between distributions, and adds up the calculated values to obtain the fifth index value. Alternatively, the server 1 may calculate, as the fifth index value, the difference between a basic statistic (e.g., median) indicating a distribution of the periodic components of the parameter and a basic statistic indicating a distribution of the periodic components of the activity amount.

As a further alternative, as illustrated in FIG. 11, the server 1 may calculate the fifth index value by directly comparing time-series data on the periodic components of the parameter with time-series data on the periodic components of the activity amount. Specifically, the server 1 separates each of the parameter and the activity amount into trend components, periodic components, and residual components, and then extracts periodic components of each of the parameter and the activity amount at each time point included in a predetermined time width. Then, the server 1 calculates, as the fifth index value, a correlation coefficient of cross-correlation between the periodic components of the parameter and the periodic components of the activity amount based on the extracted periodic components of the parameter and the activity amount. Note that when the periodic components are components based on a daily cycle, the predetermined time width refers to one day (i.e., 24 hours), for example. The predetermined time width may be, for example, a time width shorter than one day, such as half a day (i.e., 12 hours) from 6:00 AM to 6:00 PM, or a time width of two or more days.

As described above, the server 1 calculates the first to fifth index values as index values related to exacerbation of heart failure. The server 1 may calculate all of the first to fifth index values, or may calculate only some of the index values. Accordingly, it is possible to provide an index related to exacerbation of heart failure to a medical professional or the like.

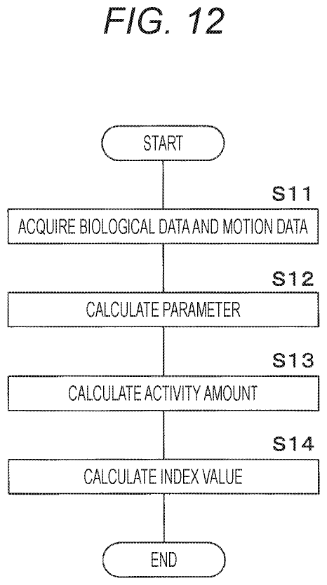

FIG. 12 is a flowchart illustrating an example of a process executed by the server 1. The content of the process executed by the server 1 will be described with reference to FIG. 12.

The control unit 11 of the server 1 acquires from the wearable device 2 biological data and motion data of a subject at each time point in a predetermined period (step S11). The biological data is a PPG signal, for example. The motion data is acceleration, for example.

The control unit 11 calculates a parameter related to the heart of the subject at each time point based on the acquired biological data (step S12). For example, the control unit 11 calculates a pulse rate as the parameter. In addition, the control unit 11 calculates the activity amount of the subject at each time point based on the acquired motion data (step S13). For example, the control unit 11 counts, as activity intensity, the number of times the numerical value calculated with Expression (1) exceeds a predetermined value within a predetermined time over time, and calculates, as the activity amount, the moving average of the activity intensity obtained as time-series data within a predetermined time window.

The control unit 11 calculates an index value related to exacerbation of heart failure based on the calculated parameter and activity amount (step S14). Specifically, the control unit 11 calculates, as with the above-described first to fourth index values, an index value related to exacerbation of heart failure based on the relationship between the parameter and a time point or a time slot where a value related to the activity amount is greater than or equal to a given value. In such a case, the control unit 11 calculates, as with the above-described first to third index values, an index value related to exacerbation of heart failure based on a distribution of the parameter with respect to the activity amount at an active time point when the activity amount is greater than or equal to a given value. Further, the control unit 11 calculates, as with the first and second index values, an index value based on the activity amount at an active time point and a parameter at a time point when a predetermined time has elapsed from a measurement time point of the activity amount.

For example, the control unit 11 calculates the first index value by comparing distribution information on a distribution of the parameter at a time point when a predetermined time has elapsed from a measurement time point of the activity amount at an active time point in the past period (i.e., first period) with distribution information on a distribution of the parameter at a time point when a predetermined time has elapsed from a measurement time point of the activity amount at an active time point in the most recent period (i.e., second period). Alternatively, the control unit 11 may calculate, as the first index value, a statistic (i.e., first statistic) indicating a distribution of the parameter at a time point when a predetermined time has elapsed from a measurement time point of the activity amount at an active time point.

In addition, for example, the control unit 11 derives, for the most recent period, a regression line indicating the relationship between the activity amount at an active time point and a parameter at a time point when a predetermined time has elapsed from a measurement time point of the activity amount, and calculates the second index value based on the slope of the regression line.

Further, for example, the control unit 11 calculates, for each of the past period (i.e., first period) and the most recent period (i.e., second period), a difference value between the parameter at each time point and the moving average of the parameter, and calculates the third index value by comparing difference value distribution information on a distribution of the difference value at an active time point in the past period with difference value distribution information on a distribution of the difference value at an active time point in the most recent period. Alternatively, the control unit 11 may calculate, as the third index value, a statistic (i.e., second statistic) indicating a distribution of the difference value at an active time point.

Furthermore, for example, the control unit 11 identifies a distribution of the activity amount for each time slot, and identifies a time slot for which a statistic (e.g., median) indicating a distribution of the activity amount is greater than or equal to a given value from the identified distribution for each time slot, and then calculates, as the fourth index value, a statistic related to a distribution of the parameter for the identified time slot.

Moreover, for example, the control unit 11 calculates the fifth index value by separating, for each of the past period (i.e., first period) and the most recent period (i.e., second period), each of the parameter and the activity amount into trend components, periodic components, and residual components, and extracting periodic components for the same segment from each of the periodic components of the parameter and the activity amount, and then comparing the extracted periodic components of the parameter and the activity amount between the past period and the most recent period. Alternatively, the control unit 11 may separate each of the parameter and the activity amount into trend components, periodic components, and residual components, and extract periodic components of each of the parameter and the activity amount at each time point included in a predetermined time width, and then calculate, as the fifth index value, a correlation coefficient of cross-correlation between the extracted periodic components of the parameter and the extracted periodic components of the activity amount.

The control unit 11 may calculate all of the first to fifth index values, or may calculate only some of the index values. The control unit 11 ends the series of process.

Although the above description illustrates an example in which the server 1 in the cloud analyzes biological data and motion data to calculate the index values, the index values may be calculated by the local wearable device 2.

As described above, according to the present embodiment, it is possible to calculate index values related to exacerbation of heart failure, and provide the index values to a medical professional or the like.

The configuration according to the present embodiment constitutes an improvement to existing computer technology for analyzing time-series data. As described above, since a subject with chronic heart failure has low heart function, the parameter (e.g., heart rate) does not increase significantly immediately after vigorous movement and does not decrease easily. Furthermore, the influence of a long-term trend of the parameter may make it difficult to distinguish the influence of the activity amount. To address these issues, the system does not merely use the parameter at the active time point, but uses the parameter at a time point when a predetermined time has elapsed from the active time point. In addition, by calculating a difference value between the parameter and a moving average of the parameter, the system eliminates the influence of the long-term trend. This specific information processing enables the server 1 to accurately calculate the index value based on the relationship between the activity amount and the parameter, thereby enhancing the analytical capability of the determination system.

### (Modification)

The above-described embodiment illustrates an aspect in which index values are calculated only from data of a subject. However, the present disclosure is not limited thereto, and the index values may be calculated by comparing data of a subject with data of a third party other than the subject (hereinafter referred to as a “reference subject”).

For example, in the above-described embodiment, each of the first, third, and fifth index values is calculated by comparing data for the past period (i.e., first period) with data for the most recent period (i.e., second period). In the present modification, data of a reference subject other than the subject is used as the data for the past period.

That is, the server 1 calculates the parameter (i.e., pulse rate) and the activity amount from biological data and motion data of the subject for the most recent period, and also acquires reference information including the activity amount of one or more reference subjects other than the subject (hereinafter referred to as “reference activity amount”) at each time point (hereinafter referred to as “reference time point”) in the past period (hereinafter referred to as a “reference period”) and a parameter related to the heart of the one or more reference subjects (hereinafter referred to as a “reference parameter”). For example, the server 1 stores in the auxiliary storage unit 14 reference information constructed based on parameters and activity amounts of persons other than the subject, and reads the reference information from the auxiliary storage unit 14.

The server 1 calculates an index value related to exacerbation of heart failure by comparing target information based on the parameter and the activity amount of the subject for the most recent period with the above-described reference information. For example, when calculating the first index value as the index value, the server 1 calculates the first index value by comparing parameter distribution information regarding the parameter at a time point when a predetermined time has elapsed from a measurement time point of the activity amount of the subject at an active time point in the most recent period with reference parameter distribution information regarding the reference parameter at a time point when a predetermined time has elapsed from a measurement time point of the reference activity amount of the reference subject at an active time point in the past period. Similarly, when calculating each of the third and fifth index values, the server 1 calculates the index value by comparing the target information on the subject with the reference information on the reference subject.

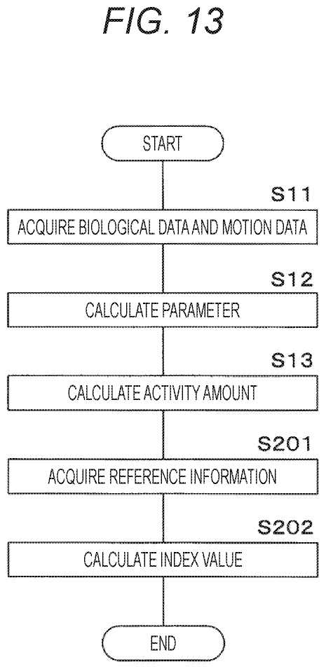

FIG. 13 is a flowchart illustrating an example of a process executed by the server 1 according to the modification. After calculating a parameter related to the heart of the subject (step S12) and calculating the activity amount of the subject (step S13), the server 1 executes the following process.

The control unit 11 of the server 1 acquires reference information obtained based on the reference activity amount of the reference subject other than the subject at each reference time point in the reference period and the reference parameter related to the heart (step S201). The control unit 11 calculates an index value related to exacerbation of heart failure from the result of comparing the target information, which is based on the parameter and the activity amount of the subject for the most recent period, with the reference information acquired in step S201 (step S202). For example, the control unit 11 calculates the first, third, and/or fifth index values by using the reference information on the reference subject as the data for the past period. The control unit 11 ends the series of processes.

Note that the auxiliary storage unit 14 stores, as reference data, parameters and activity amounts of reference subjects other than the subject, and in step S201, the control unit 11 of the server 1 may read the reference data from the auxiliary storage unit 14 and construct reference information based on the read reference data to acquire the reference information. Specifically, for example, when calculating the first index value as the index value, the control unit 11 of the server 1 constructs reference parameter distribution information from the reference data read from the auxiliary storage unit 14 in step S201, and calculates the first index value from the result of comparing the distribution information, which is based on the parameter and the activity amount of the subject for the most recent period, with the constructed distribution information on the reference subject in step S202.

As described above, according to the present modification, the index value can also be calculated using data of the reference subject other than the subject.

It should be construed that the embodiments disclosed herein are illustrative in all respects and not restrictive. The scope of the present disclosure is defined by the claims rather than the above description and is intended to include all modifications within the meaning and scope of the claims.

Some or all of the subject matters described in the respective embodiments can be combined together. In addition, some or all of the independent claims and their dependent claims described in the claims can be combined together, regardless of their dependent relationships. Furthermore, although a format (multiple dependent claim format) in which a claim depends on two or more other claims is described is used in the claims, the claim form is not limited thereto. The claims may be described in a format (multiple multiple dependent claim) in which a multiple dependent claim depends on at least one multiple dependent claim.

What is claimed is:

1. A system for determining heart failure exacerbation, comprising:
a wearable device including one or more sensors configured to measure biological data and motion data of a subject; and
a server connectable to the wearable device and including a processor and a memory storing a program that, when executed by the processor, causes the server to:
acquire, from the wearable device, the biological data and the motion data at each time point in a predetermined period,
calculate a parameter related to a heart at each time point based on the biological data,
calculate an activity amount of the subject at each time point based on the motion data,
identify active time points among time points in the predetermined period where the calculated activity amounts are equal to or greater than a predetermined value,
identify specific parameters at delayed time points when a predetermined time has elapsed from the identified active time points, and
calculate an index value related to the heart failure exacerbation based on a relationship between the activity amounts at the active time points and the specific parameters at the delayed time points.

2. The system according to claim 1, wherein the program causes the server to calculate the index value based on a distribution of the specific parameters with respect to the activity amounts.

3. The system according to claim 1, wherein the program causes the server to:
calculate the parameter and the activity amount by acquiring the biological data and the motion data for each of a first period and a second period, and
calculate the index value by comparing distribution information on a distribution of the specific parameters in the first period with distribution information on a distribution of the specific parameters in the second period.

4. The system according to claim 1, wherein the program causes the server to calculate, as the index value, a first statistic indicating a distribution of the specific parameters.

5. The system according to claim 1, wherein the program causes the server to:
calculate the parameter and the activity amount by acquiring the biological data and the motion data of the subject for a most recent period,
derive, for the most recent period, a regression line indicating a relationship between the activity amounts at the active time points and the specific parameters, and
calculate the index value based on a slope of the regression line.

6. The system according to claim 1, wherein the program causes the server to:
calculate the parameter and the activity amount by acquiring the biological data and the motion data for each of a first period and a second period,
for each of the first and second periods, calculate a difference value between the parameter calculated at each time point and a moving average of the parameter, and
calculate another index value by comparing information on a distribution of the difference values at the active time points in the first period with information on a distribution of the difference values at the active time points in the second period.

7. The system according to claim 1, wherein the program causes the server to:
calculate a difference value between the parameter calculated at each time point and a moving average of the parameter, and
calculate, as another index value, a second statistic indicating a distribution of the difference values at the active time points.

8. The system according to claim 1, wherein the program causes the server to:
identify, from a distribution of the activity amounts for each time slot, a time slot for which a statistic indicating the distribution of the activity amounts is greater than or equal to a predetermined value, and
calculate, as another index value, a statistic related to a distribution of the parameters included in the identified time slot.

9. The system according to claim 1, wherein the program causes the server to:
calculate the parameter and the activity amount by acquiring the biological data and the motion data for each of a first period and a second period,
separate, for each of the first and second periods, each of the parameter calculated at each time point and the activity amount calculated at each time point into trend components, periodic components, and residual components,
extract, for each of the first and second periods, periodic components for a same segment from the separated periodic components of each of the parameter and the activity amount, and
calculate another index value by comparing the extracted periodic components of the parameter and the activity amount between the first and second periods.

10. The system according to claim 1, wherein the program causes the server to:
separate each of the parameter calculated at each time point and the activity amount calculated at each time point into trend components, periodic components, and residual components,
extract periodic components of each of the parameter and the activity amount included in a predetermined time interval from the separated periodic components, and
calculate another index value based on a correlation coefficient of cross-correlation between the extracted periodic components of the parameter and the extracted periodic components of the activity amount.

11. The system according to claim 1, wherein the program causes the server to:
calculate the parameter and the activity amount by acquiring the biological data and the motion data of the subject for a most recent period,
acquire reference information obtained based on reference activity amounts of a reference subject at each reference time point in a reference period and reference parameters related to a heart of the reference subject, and
calculate an index value from a result of comparing target information that is based on the specific parameters and the activity amounts for the most recent period with the reference information.

12. A non-transitory computer-readable storage medium storing a program for determining heart failure exacerbation, the program causing a computer to execute a method comprising:
acquiring, from a wearable device, biological data and motion data of a subject at each time point in a predetermined period;
calculating a parameter related to a heart at each time point based on the biological data;
calculating an activity amount of the subject at each time point based on the motion data;
identifying active time points among time points in the predetermined period where the calculated activity amounts are equal to or greater than a predetermined value;
identifying specific parameters at delayed time points when a predetermined time has elapsed from the identified active time points; and
calculating an index value related to the heart failure exacerbation based on a relationship between the activity amounts at the active time points and the specific parameters at the delayed time points.

13. The non-transitory computer-readable storage medium according to claim 12, wherein the index value is calculated based on a distribution of the specific parameters with respect to the activity amounts.

14. The non-transitory computer-readable storage medium according to claim 12, wherein
the parameter and the activity amount are calculated from the biological data and the motion data acquired for each of a first period and a second period, and
the index value is calculated by comparing distribution information on a distribution of the specific parameters in the first period with distribution information on a distribution of the specific parameters in the second period.

15. The non-transitory computer-readable storage medium according to claim 12, wherein the method further comprises:
calculating, as the index value, a first statistic indicating a distribution of the specific parameters.

16. The non-transitory computer-readable storage medium according to claim 12, wherein
the parameter and the activity amount are calculated from the biological data and the motion data of the subject acquired for a most recent period,
the method further comprises deriving, for the most recent period, a regression line indicating a relationship between the activity amounts at the active time points and the specific parameters, and
the index value is calculated based on a slope of the regression line.

17. The non-transitory computer-readable storage medium according to claim 12, wherein
the parameter and the activity amount are calculated from the biological data and the motion data acquired for each of a first period and a second period, and
the method further comprises:
for each of the first and second periods, calculating a difference value between the parameter calculated at each time point and a moving average of the parameter; and
calculating another index value by comparing information on a distribution of the difference values at the active time points in the first period with information on a distribution of the difference values at the active time points in the second period.

18. The non-transitory computer-readable storage medium according to claim 12, wherein the method further comprises:
calculating a difference value between the parameter calculated at each time point and a moving average of the parameter; and
calculating, as another index value, a second statistic indicating a distribution of the difference values at the active time points.

19. The non-transitory computer-readable storage medium according to claim 12, wherein the method further comprises:
identifying, from a distribution of the activity amounts for each time slot, a time slot for which a statistic indicating the distribution of the activity amounts is greater than or equal to a predetermined value; and
calculating, as another index value, a statistic related to a distribution of the parameters included in the identified time slot.

20. An apparatus for determining heart failure exacerbation, comprising:
a network interface circuit connectable to a wearable device;
a memory; and
a processor configured to execute a program stored in the memory to:
acquire, from the wearable device, biological data and motion data of a subject at each time point in a predetermined period,
calculate a parameter related to a heart at each time point based on the biological data,
calculate an activity amount of the subject at each time point based on the motion data,
identify active time points among time points in the predetermined period where the calculated activity amounts are equal to or greater than a predetermined value,
identify specific parameters at delayed time points when a predetermined time has elapsed from the identified active time points, and
calculate an index value related to the heart failure exacerbation based on a relationship between the activity amounts at the active time points and the specific parameters at the delayed time points.
